(19) United States
(12) Patent Application Publication
(10) Pub. No.: US 2019/0388548 A1
(43) Pub. Date: Dec. 26, 2019

(54) METHOD FOR PROVIDING OCULAR NEUROPROTECTION OR FOR PREVENTING, TREATING OR ALLEVIATING THE EFFECTS OF, AN OCULAR DISEASE ASSOCIATED WITH RETINAL GANGLION CELL DEATH

(71) Applicant: TZU CHI UNIVERSITY, Hualien (TW)

(72) Inventors: Shun-Ping Huang, Hualien (TW); Rong-Kung Tsai, Hualien (TW)

(73) Assignee: TZU CHI UNIVERSITY, Hualien (TW)

(21) Appl. No.: 16/019,493

(22) Filed: Jun. 26, 2018

(51) Int. Cl. A61K 47/64; A61K 39/395; A61P 27/02

(52) U.S. Cl. CPC A61K 47/64; A61P 27/02; A61K 39/39541

(57) ABSTRACT

The present invention relates to a method for providing ocular neuroprotection or for preventing, treating or alleviating the effects of, an ocular disease associated with retinal ganglion cell death in a subject in need thereof, comprising administering to said subject an effective amount of a recombinant P-selectin immunoglobin G (P-sel-IgG) chimeric fusion protein, or a composition comprising the protein and a pharmaceutically acceptable adjuvant, vehicle, or carrier.

### FIELD OF THE INVENTION

The present invention relates to a method for providing ocular neuroprotection or for preventing, treating or alleviating the effects of, an ocular disease associated with retinal ganglion cell death in a subject in need thereof, comprising administering to said subject an effective amount of a recombinant P-selectin immunoglobin G (P-sel-IgG) chimeric fusion protein, or a composition comprising the protein and a pharmaceutically acceptable adjuvant, vehicle, or carrier.

### BACKGROUND OF THE INVENTION

Retinal ischemia, which leads to profound vision loss, is a common pathology in many eye disorders, including ischemic optic neuropathies, diabetic retinopathy, retinal artery occlusion, choroidal neovascularization (CNV) and glaucoma. Retinal ischemia involves reduced oxygen, metabolites and waste product clearance. Damage to the retina, an extension of the central nervous system (CNS), is irreversible and can result in the death of retinal ganglion cells (RGCs), amacrine cells, and bipolar cells, depending on the disease type and status. Retinal ischemia induced-optic disc drusen (crowded optic nerve), impaired retinal vasculature, hemorrhage, neovascularization, and retinal detachment cause vision loss. The pathophysiology aspects of retinal ischemic diseases have been studied previously and various mechanisms have been hypothesized. Disease mechanisms that may lead to cell death are oxidative stress in the retina, expression of pro-inflammatory factors in the optic nerve, disruption of calcium ion homeostasis, and macrophage polarization. Considering these mechanisms, some strategies can reduce tissue damage with anti-inflammatory compounds, neurotropic factors, oxidative stress regulators, calcium channel blockers and microglial activation inhibitors or blood-borne macrophage infiltration blockers. The rat anterior ischemic optic neuropathy (rAION) model represents an excellent model to investigate RGC pathology and ischemic injury because rAION shares similar features and pathology with human and primate AION.

The rAION model achieved by photodynamic therapy will generate superoxide radicals that circulate within optic nerve (ON) capillaries, causing ON infarct and ischemia. Inflammation and oxidative stress generated by reactive oxygen species (ROS) in rAION cause RGC death. Therefore, reducing this inflammatory response and oxidative stress can prevent RGC apoptosis.

P-selectin (CD62), a member of the selectin family, is confined to the α-granules of platelets and Weibel-Palade bodies of endothelial cells. P-selectin is translocated to the surface upon activation of endothelial cells or platelets for leukocyte recruitment. The P-selecting PSGL-1 (P-selectin glycoprotein ligand-1) interaction supports leukocyte rolling and firm adhesion, leading to transmigration in surrounding tissue that triggers an inflammatory response cascade. A soluble recombinant form of exogenous P-selectin can restore hemostasis in a mouse model of hemophilia, rescue viper venom-induced mortality, rescue liver endothelial cells from ischemic reperfusion injury and ameliorate inflammation. All these findings are based on one common principle; the soluble recombinant form of exogenous P-selectin competes with endogenous membrane bound P-selectin molecules to bind with PSGL-1, a well-known ligand for P-selectin. Although there is similar pathophysiology in rAION, including ischemia, photothrombosis, and inflammation, the therapeutic potential of soluble P-selectin in ischemic injury still needs to be further investigated. In addition, stopping the inflammatory process is a potential therapeutic target, but little is known about the antioxidative pathway in rAION. Oxidative stress caused by the production of ROS triggers a stress response via the nuclear factor erythroid 2-related factor 2 (Nrf2)-antioxidant response element (ARE) signaling axis, which scavenges ROS and maintains redox status. It was thought that Nrf2 was limited to redox control and that antiinflammatory effects were the result of the elimination of ROS by Nrf2. However, Nrf2 inhibits the transcription of proinflammatory cytokines by binding in close proximity to these genes in ARE-dependent manner. Therefore, the antioxidant pathway as an inflammatory counterpart in rAION still needs to be further explored.

### BRIEF DESCRIPTION OF THE DRAWINGS

FIG. 1: FVEPs. (a) Representative FVEP profile after rAION in each group (the box indicates the P1-N2 amplitude). (b) Bar charts showing the P1-N2 amplitude. The amplitudes of the 4 μg P-sel- and 2 μg P-sel-treated groups were significantly higher than those of the PBS-treated group (25.16571±7.931084 μV and 16.296±5.484773 μV, respectively). Data are expressed as the mean±S.D.; *P≤0.05, **P≤0.1 n=6.

FIG. 2: Retinal flat mount preparations and RGC morphometry. (a,b) Representative image of RGC density after rAION in each group. The 4 μg P-sel-treated group showed significantly higher RGC density than the PBS-treated group in the (c) central (1009±177/mm² versus 612±31/mm², respectively) and (d) mid-peripheral retina (614±99/mm² versus 323±92/mm², respectively). The 2 μg P-sel-treated group also showed significantly higher RGC density than the PBS-treated group in the mid-peripheral retina (d) (544±66/mm² versus 323±92/mm², respectively). **P≤0.01, ***P≤0.001; n=6.

FIG. 3: TUNEL assay in the retina. (a) Representative images of TUNEL-stained retinal cross sections after rAION in each group. (b) The 4 μg P-sel-treated group showed significantly fewer TUNEL+ cells than the PBS-treated group in the central retina (13.30±6.290717706 versus 24.5±8.06, respectively). GCL, ganglion cell layer; IPL, inner plexiform layer; INL, inner nuclear layer; OPL, outer plexiform layer; ONL, outer nuclear layer; *P≤0.05, ***P≤0.001; n=6.

FIG. 4: ED1 immunostaining of the ON. (a) Representative images of ED1 immunostaining in ON cross-sections after rAION in each group. (b) The 4 μg P-sel- and 2 μg P-sel treated groups showed significantly fewer ED1+ cells than the PBS-treated group (16.53±10.26 and 20.2±10.29 versus 36.5±11.3, respectively). **P≤0.01, ***P≤0.001; n=6.

FIG. 5: OCT profile of RNFL and ONW. (a) Linear scan across the optic nerve head. (b-d) Representative ONW profiles of the sham, rAION and 4 μg P-sel-treated groups at day 3. (e) ONW thickness profile over time. Compared with the PBS-treated group, the 4 μg P-sel-treated group exhibited a significant reduction in edema (385.25±48 μm versus 325.5±37.3 μm, respectively). (f) Circular scan around the optic nerve head. (g-i) Representative RNFL thickness measurement of the sham, rAION and 4 μg P-sel-treated groups at day 28 (the black line indicates the RNFL). (j-l) Representative ONW profile of the sham, rAION and 4 μg P-sel-treated groups at day 28. (m) RNFL thickness profile (area under the curve) over time. Compared with the PBS-treated group, the 4 μg P-sel-treated group exhibited significant preservation of the RNFL at day 28 (0.5±0.15 mm² versus 0.68±0.17 mm², respectively). RNFL, retinal nerve fiber layer; GCL, ganglion cell layer; IPL, inner plexiform layer; INL, inner nuclear layer; OPL, outer plexiform layer; *P≤0.05; n=6.

FIG. 6: TEM of optic nerve cross sections. (a) Pictorial representation of the neurovascular unit with its major components (red blood cell, RBC; basal lamina, BL; neurons, N; astrocyte end feet, AE; endothelial cell, EC; pericyte, P). (b) Cross-section image of a capillary of a sham. Intact ultrastructure with distinguishable components of the neurovascular unit; (n=1). (c) Inset with prominent tight junctions (black arrows). (d,h) Blood-optic nerve barrier (BONB) disruption with all components missing and (e,i) severe vacuolation in the BONB at day 1 and day 7 in the PBS-treated group; (n=2). (f, j) Preserved BONB with visible tight junctions (inset (g, k), black arrows) in the 4 μg P-sel-treated group at day 1 and day 3; (n=2). (l) Reconstitution of the BONB at day 7 in the PBS-treated group. (m) Inset showing tight junctions; (n=1). (n) The BONB of the 4 μg P-sel-treated group at day 7. (o) Inset showing tight junctions.

FIG. 7: Immunoblots of the retina. (a) Representative cropped blot images of Nrf2, NQO1, and GAPDH (internal loading control). (b,c) Bar charts showing the relative density of Nrf2, HO-1 and Nqol with a sham retina as a reference. *P≤0.05, **P≤0.01, ***P≤0.001; n=3.

FIG. 8: Summary of this study (d) and a possible model for the neuroprotective effect of P-selectin-IgG in the rAION model. P-sel-IgG treatment after rAION induction (a) can saturate Psgl-1 (b, inset c) and stop macrophage infiltration in optic nerve tissue.

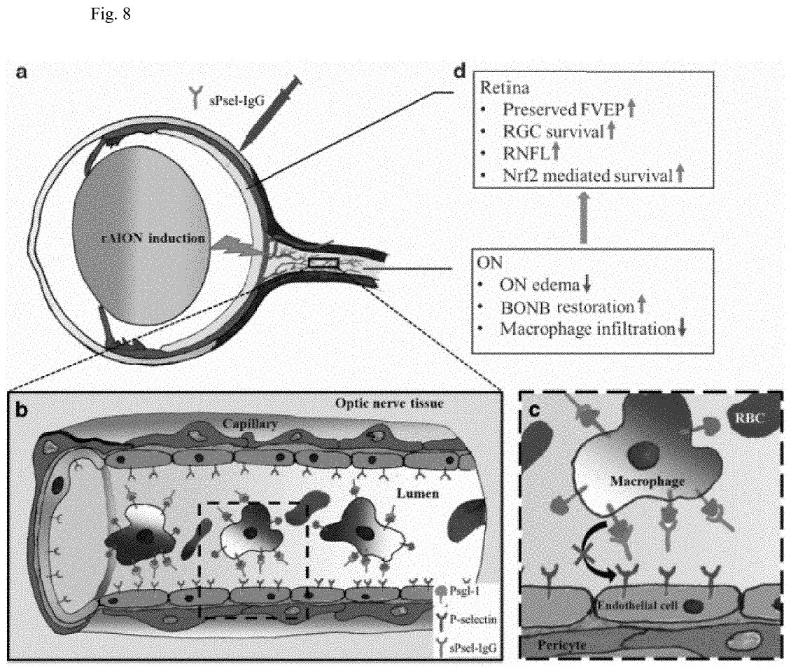

### SUMMARY OF THE INVENTION

The present invention relates to a method for providing ocular neuroprotection or for preventing, treating or alleviating the effects of, an ocular disease associated with retinal ganglion cell death in a subject in need thereof, comprising administering to said subject an effective amount of a recombinant P-selectin immunoglobin G (P-sel-IgG) chimeric fusion protein, or a composition comprising the protein and a pharmaceutically acceptable adjuvant, vehicle, or carrier.

### DETAILED DESCRIPTION OF THE INVENTION

The present invention demonstrates the neuroprotective effect of a recombinant P-selectin immunoglobin G (P-sel-IgG) chimeric fusion protein in a rat anterior ischemic optic neuropathy (rAION) model. Assuming that P-sel-IgG will bind to PSGL-1, the present study also examines the mechanism by which P-sel-IgG affects visual function, RGC survival, the blood-optic nerve barrier (BONB) and leukocyte recruitment after ischemic injury. rAION was induced by photodynamic therapy. P-sel-IgG treatment reduces optic nerve edema and stabilizes the blood-optic nerve barrier (BONB) in the acute phase of rAION. Further, P-sel-IgG increases the retinal ganglion cell (RGC) survival rate, reduces RGC apoptosis, preserves visual function, maintains retinal nerve fiber layer thickness, and reduces macrophage infiltration in optic nerve tissue in the chronic phase (day 28). Increased NAD(P)H quinone dehydrogenase 1 (NQO1) and heme oxygenase 1 (HO-1) expression levels, along with increased transcription factor Nrf2, suggesting an antioxidant role of P-sel-IgG via the Nrf2 signaling pathway. In conclusion, this study is the first to demonstrate that P-sel-IgG treatment promotes RGC survival by stabilizing the BONB and activating the Nrf2 signaling pathway in a rAION model. P-sel-IgG would be a potential therapeutic application for the treatment of ischemic ON and retinal vascular diseases. Since the ON is part of the CNS, and AION pathology is similar to other types of stoke in the CNS, P-sel-IgG treatment may also be effective for treatment of other types of CNS strokes or white matter ischemia.

Therefore, the present invention provides a method for providing ocular neuroprotection or for preventing, treating or alleviating the effects of, an ocular disease associated with retinal ganglion cell death in a subject in need thereof, comprising administering to said subject an effective amount of a recombinant P-selectin immunoglobin G (P-sel-IgG) chimeric fusion protein, or a composition comprising the protein and a pharmaceutically acceptable adjuvant, vehicle, or carrier. In an embodiment, the ocular disease comprises visual field loss. In an embodiment, the ocular disease comprises neurodegeneration, increased intraocular pressure, an ischemic event or optic nerve injury. In an embodiment, the ocular disease comprises injury to the retina or optic nerve injury, in which the injury to the retina or optic nerve injury comprises ischemia or hypoxia injury. In an embodiment, the ocular disease is selected from the group consisting of glaucoma, diabetic retinopathy (DR), diabetic macular edema (DME), age related macular degeneration (AMD), Leber's hereditary optic neuropathy (LHON), Leber optic atrophy, optic neuritis, retinal artery occlusion, central retinal vein occlusion, branch retinal vein occlusion, ischemic optic neuropathy, optic nerve injury, retinopathy of prematurity (ROP) or retinitis pigmentosa (RP), retinal ganglion degeneration, macular degeneration, hereditary optic neuropathy, metabolic optic neuropathy, optic neuropathy due to a toxic agent, neuropathy caused by adverse drug reactions or vitamin deficiency, and vision loss associated with a tumor. In an embodiment, the ocular disease is ischemic optic neuropathy. In an embodiment, the ischemic optic neuropathy is anterior ischemic optic neuropathy (AION). In an embodiment, the ocular neuroprotection comprises neuroprotection of the optic nerve.

In the above method, the protein or the composition comprising the protein is administered as a cream, a foam, a paste, an ointment, an emulsion, a liquid solution, an eye drop, a gel, spray, a suspension, a microemulsion, microspheres, microcapsules, nanospheres, nanoparticles, lipid vesicles, liposomes, polymeric vesicles, a patch, or a contact lens. In an embodiment, the protein or the composition comprising the protein is administered as a liquid solution, which is administered by intravitreal injection.

In the above method, the protein comprises a C-type lectin domain and an EGF-like domain of P-selectin fused with the Fc region of human IgG₁ in a disulfide-linked homodimer form.

### EXAMPLES

The examples below are non-limiting and are merely representative of various aspects and features of the present invention.

### Example 1

### Materials and Methods

A list of resources used in this study was provided in Table 1.

TABLE 1
List of resources used in this study

| | | |
|---|---|---|
| Reagent or resource | Source | Identifier |

Antibodies and recombinant proteins

| | | |
|---|---|---|
| Goat anti-mouse | Life Technologies OR, | Cat#A11001 |
| Alexa 488 | USA | Lot# 1613346 |
| Goat anti-mouse | Bio-Rad Laboratories, | Cat#170-6516 |
| HRP | Inc., CA, USA | |
| Goat anti-rabbit | Jackson Immuno | Cat#111-035-00, |
| HRP | research | Lot# 126526 |
| | laboratories, Inc., | |
| | PA, USA | |
| Mouse monoclonal | Bio-Rad Laboratories, | Cat#MCA341GA |
| anti-CD68 | Inc., CA, USA | |
| Mouse monoclonal | Sigma-Aldrich co., MO, | CAT# G8795 |
| anti-GAPDH | USA | |
| Mouse monoclonal | Santa Cruz | Cat# sc-32793, |
| anti-NQO1 | Biotechnology, | Lot# K2816 |
| | Inc., USA | |
| Rabbit polyclonal | Abcam, MA, USA | Ca#-ab13243 |
| anti-HO-1 | | |
| Rabbit polyclonal | Santa Cruz | Cat# sc-722, |
| anti-Nrf2 | Biotechnology, | Lot# I2211 |
| | Inc., USA | |
| Recombinant Mouse | R&D Systems, Inc. MN | Cat# 737-PS, |
| P-Selectin/CD62P | | Lot# DIF0814121 |
| Fc Chimera Protein | | |

Commercial assays

| | | |
|---|---|---|
| Protein BCA kit | Thermo Scientific, IL, | Cat# 23225 |
| | USA | Lot# OA183168 |
| TUNEL assay | Promega Corporation, | Cat#G3250, |
| | WI, USA | Lot#0000180289 |

Animal model

| | | |
|---|---|---|
| Outbred male | BioLASCO Taiwan Co., | N/A |
| Wistar rats | Ltd., Taiwan | |

Equipment

| | | |
|---|---|---|
| Chemiluminescence | UVP, LLC, CA, USA | Cat# BioSpectrum |
| Western blot | | 810, N/A |
| imaging | | |
| Cryostat | Leica Microsystems, | Cat# Leica |
| (cryosectioning) | Germany | CM3050S, N/A |
| Fluorescence | Carl Zeiss Meditech Inc., | Cat# Axioplan 2 |
| microscope | Thornwood, NY, USA | imaging, N/A |
| FVEP stimulator | Diagnosys LLC, MA, | Cat# Colordome |
| | USA | ganzfeld, N/A |
| Green Laser | NIDEK CO., LTD, Japan | Cat# GYC-500, |
| Photocoagulator | | N/A |
| Spectral domain | Phoenix research labs, | Cat# Micron IV, |
| OCT | CA, USA | N/A |
| Transmission | Hitachi High-Technologies | Cat# Hitachi |
| electron | Corporation, japan | H-7500, N/A |
| microscope | | |
| Ultramicrotome | Leica Microsystems, | Cat# Leica EM |
| | Germany | UC6, N/A |

CHEMICALS pharmaceutical grade

| | | |
|---|---|---|
| Balanzine(Xylazine | Health-Tech | Cat# Balanzine |
| 2% w/v) | Pharmaceutical Co., | 2%, Lot# 502001 |
| | Taipei, Taiwan | |
| Fluoro-gold | Flurochrome LLC, Denver, | N/A |
| | CO, USA) | |
| Imalgene | Merial, France | Cat# Imalgene |
| 1000(ketamine | | 1000, |
| 100 mg/ml) | | Lot# LBM155AA |
| Phenylephrine | Santen Pharmaceutical, | Cat# Mydrin-P, |
| hydrochloride | Osaka, Japan | Lot#mp2010 |
| eye drops | | |
| Proparacaine | Alcon-Couvreur, N.V., | Cat# Alcaine, |
| Hydrochloride | Puurs, Belgium | Lot#16e26ed |
| Ophthalmic | | |
| Solution | | |
| Rose bengal | Sigma-Aldrich Co., MO, | Cat# R4507, N/A |
| | USA | |
| Tobramycin, | Alcon-Couvreur, N.V., | Cat# Tobradex, |
| Dexamethasone | Puurs, Belgium | Lot# 13J30K |

Reagents, Buffers, and solutions

| | | |
|---|---|---|
| Bis-acrylaminde | Bio-Rad Laboratories, | Cat# 161-0156, |
| | Inc., CA, USA | N/A |
| FBS | Gibco life technologies, | Cat#26140-079 |
| | USA | |
| Glutaraldehyde | Electron microscopy | Cat# 16220, N/A |
| | sciences, PA, USA | |
| Immobilon-P^{SQ}(PVDF | Millipore corporation, | Cat# ISEQ00010, |
| membrane) | MA, USA | Lot# K2MA7796H |
| Methanol | Avantor performance | Cat# 9093-68, |
| | materials. Inc. PA, USA | Lot# 0000067375 |
| Osmium tetroxide | Electron microscopy | Cat# 19190, N/A |
| | Sciences, PA, USA | |
| PBS | Gibco life technologies, | Cat#70011-044 |
| | USA | |
| Sodium cacodylate | Electron microscopy | Cat# 12300, N/A |
| | sciences, PA, USA | |
| Spurr's resin | Electron microscopy | Cat# 14300, N/A |
| | sciences, PA, USA | |
| Uranyl acetate | Electron microscopy | N/A, N/A |
| | sciences, PA, USA | |

Software and algorithms

| | | |
|---|---|---|
| AMT camera system | Advanced Microscopy | N/A |
| | Techniques, Corp., MA, | |
| | USA | |
| Axiovision LE | Carl Zeiss micro imaging | N/A |
| Discover OCT | Phoenix research labs, CA, | N/A |
| | USA | |
| Espion V6 | Diagnosys LLC, MA, | N/A |
| | USA | |
| Image j | https://imagej.nih.gov/ij/ | N/A |
| Image Master 2D | GE Healthcare | N/A |
| Platinum | Bio-Sciences, Sweden | |
| Insight | Phoenix research labs, CA, | N/A |
| | USA | |

### Animals:

Sixty-one outbred adult Wistar rats weighing 150-180 grams (7-8 weeks) were maintained in filter top holding cages. The rats had free access to food and water in an environmentally controlled room at a temperature of 23° C. and 55% humidity with a 12-h light-dark cycle (light period 7 a.m.-p.m.). Animal care and experimental procedures were conducted in accordance with the ARVO statement for the use of Animals in Ophthalmic and Vision Research, and the Institutional Animal Care and Use Committee (IACUC) at the laboratory animal center, Tzu Chi University approved all the animal experiments. An intramuscular injection of a ketamine (100 mg/kg) and xylazine (10 mg/kg) cocktail was administered for general anesthesia. Alcaine was applied for local anesthesia, and Mydrin-P was applied for pupil dilation in all the experiments. Study design details are provided in Table 2.

TABLE 2
Summary of rats used in this study

| | | | rAION + | rAION + |
|---|---|---|---|---|
| Experiments | Sham | rAION | 2 μg P-sel | 4 μg P-sel |
| FG/OCT | 6 | 6 | 6 | 6 |
| VEP/OCT/Immunoblot/ | 6 | 6 | 6 | 6 |
| IHC/TUNEL | | | | |
| TEM | 1 | 6 | 0 | 6 |

| | |
|---|---|
| Total | 61 |

### AION Induction:

Alcaine and Mydrin-P eye drops were applied for local anesthesia and pupil dilation, respectively. After general anesthesia, 2.5 mM rose bengal in PBS (1 ml/kg animal weight) was intravenously administered. Immediately after rose bengal injection, the optic disc was exposed to an argon green laser (532 nm wavelength, 500 mm size and 80 mW power) for 12 l-s pulses. A fundus lens was used to focus the laser on the optic disc. Tobradex eye ointment was applied after the procedure, and the rats were monitored until complete recovery was observed.

### P-Sel-IgG Administration and Formulation:

We used recombinant mouse P-selectin-Fc chimera protein (P-sel-IgG), which comprises a C-type lectin domain and an EGF-like domain of P-selectin fused with the Fc region of human IgG₁ in a disulfide-linked homodimer form. In brief, 200 μg P-sel-IgG was reconstituted in a 200 μl PBS:glycerol (8:2) solution to achieve a 1 μg/μl concentration. The animals were either treated with PBS, 4 μg P-sel-IgG (4 μg P-sel), or 2 μg P-sel-IgG (2 μg P-sel) in a total volume of 4 μl by IVI.

### Flash Visually Evoked Potential Recordings:

After general anesthesia, the sagittal region of the skull was opened. Screw implants were fixed at the primary visual cortex region of both hemispheres using stereotaxic coordinates (AP: anterior-posterior; ML: medial-lateral; DV: dorsal-ventral; AP: −8 mm; and ML: −3.0 mm); one electrode was fixed at the frontal cortex (AP: 3 mm). FVEPs were measured using a visual electrodiagnostic system. The system had built-in programs to measure FVEPs. Electrodes at the primary visual cortex were considered active (positive) electrodes, the electrode at the frontal cortex was considered the reference (negative) electrode, and the ground electrode was placed in the rat's tail. The settings used were as follows: no background Illumination, a flash intensity of 30 cd·s/m², and a single flash with a flash rate of 1.02 Hz. An average of 64 sweeps were collected, and the raw data were saved for further analysis. The P1-N2 amplitude was measured to check visual function.

### Retrograde Labeling of RGCs by Fluoro-Gold and Measurement of RGC Density:

RGCs were labeled in a retrograde manner as described in a previous report (Huang T L, Huang S P, Chang C H, Lin K H, Sheu M M, Tsai R K. Factors influencing the retrograde labeling of retinal ganglion cells with fluorogold in an animal optic nerve crush model. Ophthal res 2014; 51: 173-178). In brief, retrograde labeling was performed 1 week before the rats were sacrificed. The sagittal region of the skull was opened, and 2 μl fluoro-gold was injected into the superior colliculus (AP: −6 mm; ML: −1.5 mm; and DV 4 mm). The same procedure was performed on the other hemisphere. One week after labeling, the rats were killed, and the eyeballs were collected and fixed in 10% formalin. Retinas were carefully flat mounted. The retina was examined under a fluorescence microscope with ×100 power, an inbuilt filter set (excitation filter, 350-400 nm; barrier filter, 515 nm) and a connected digital imaging system. The retina was examined from 1 mm to 3 mm from the center to calculate central and peripheral RGC densities. At least 10 random regions were separately scanned in the central and mid-peripheral regions; images of these cells were saved for density calculation. RGC density was calculated by ImageMaster 2D Platinum software. The RGC survival rate was determined by calculating the ratio of the treatment groups to the sham-operated group and multiplying the ratio by 100.

### Retinal and ON Sample Preparation:

The rats were killed, and their eyes were enucleated and fixed in 4% paraformaldehyde. The eyeballs and ONs were separated and transferred to 30% sucrose; the samples were stored at 4° C. until they settled at the bottom of the tubes. Retina and ON cross sections of 20 μm were obtained using a cryostat.

### ED-1 Immunohistochemistry (IHC) on ON Tissues:

Anti-ED-1 was specific for extrinsic macrophages. ON cross-sections were blocked with 5% FBS for 1 h at room temperature. The tissue was labeled with an ED1 primary antibody diluted in antibody dilution buffer (2% BSA, 1×PBS (pH 7.2), and 0.3% Triton X-100; 1:200) overnight at 4° C. Goat anti-mouse Alexa 488 (0.3% Triton X-100 and 1×PBS (pH 7.2); 1:500) was added to the tissues, which were incubated for 1 h at room temperature and counterstained with DAPI (0.3% Triton X-100 and 1×PBS (pH 7.2); 1:500). Image acquisition was conducted with appropriate filter sets in a fluorescence microscope at ×100 magnification. ED-1⁺ cell counting was manually performed or conducted by ImageMaster 2 Platinum software.

### Tunel Assay:

TUNEL was used to detect apoptotic cells in the ganglion cell layer (GCL). A TUNEL assay was performed according to the manufacturer's protocol (DeadEnd Fluorometric TUNEL System; Promega Corporation, Madison, Wis., USA). TUNEL⁺ cells in the GCL were manually counted.

### Image-Guided OCT Imaging:

A Phoenix Micron IV retinal microscope with image-guided OCT was used for imaging. This system used spectral domain OCT, which provided a longitudinal resolution of 1.8 μm and a transverse resolution of 3 μm with a 3.2-mm field of view and 1.2-mm imaging depth at the retina. After general anesthesia, the rats were placed on the imaging platform, and the head was positioned at an angle to allow the penetration of light vertical to the cornea from the temporal side. The RNFL was obtained by circular scanning around the optic disc, and the Bruch membrane opening (ONW) was scanned by a linear scan through the center of the optic disc. At least three clear captures were obtained for each eye. Quantitative measurements of the Bruch membrane opening and RNFL thickness were carried out by built-in ‘Insight’ software. This software generated a segment of different layers and a thickness profile of the desired segmented layer. The average RNFL thickness was measured by calculating the area under the curve for the RNFL thickness profile with GraphPad Prism. The above-mentioned procedure was performed at pre-rAION (day 0) and at day 1, day 3, day 7, day 14 and day 28 post-rAION.

### Transmission Electron Microscopy of ON:

The rats were killed at different time points (day 1, day 3, and day 7), and the ON tissues (1 to 2 mm³) were dissected 1 mm away from the ON head. The tissues were prefixed in 2.5% glutaraldehyde/0.1 M cacodylate buffer+1% tannic acid. The tissues were then post-fixed with 1% osmium tetroxide/0.1 M cacodylate buffer. After post-fixation, the tissues were subjected to en block staining with 2% uranyl acetate. The tissues were then embedded in Spurr's resin, and 80-nm-thick cross-sections were obtained with an ultra-microtome and observed by TEM. An average of 4-5 microphotographs of capillaries was taken per sample at the desired magnification.

### Western Blotting:

The rats were killed, and their eyes were enucleated. The retinas were homogenized and stored at −80° C. for further analysis. A protein assay was performed using a BCA protein assay kit. For immunoblotting, 30 μg of protein was separated on a 10% bis-acrylamide gel. The proteins were transferred to polyvinylidene difluoride membranes. After the transfer, the membranes were blocked with 5% non-fat dry milk for 1 h, followed by an overnight incubation with Nrf2 (1:250; Santa Cruz Biotechnology, Santa Cruz, Calif., USA), Nqol (1:500; Santa Cruz), Hol (1:1000; Abcam, Cambridge, Mass., USA), or GAPDH (1:2000; Sigma-Aldrich, St. Louis, Mo., USA) primary antibody at 4° C. The membranes were washed, followed by incubating with a secondary antibody conjugated to HRP against the appropriate host species for 1 h at room temperature. The membranes were then developed using enhanced chemiluminescent substrate, and images were taken in a western blot analyzer. The relative density was calculated using ImageJ software.

### Statistical Analysis:

All statistical analyses was performed using GraphPad Prism. The data are presented as the mean±S.D. A Mann-Whitney U-test was used for comparisons between groups. P-values less than 0.05 were considered statistically significant, with * representing P≤0.05, **P≤0.01, and ***P≤0.001.

### Results

### P-Sel-IgG Treatment Preserved Visual Function:

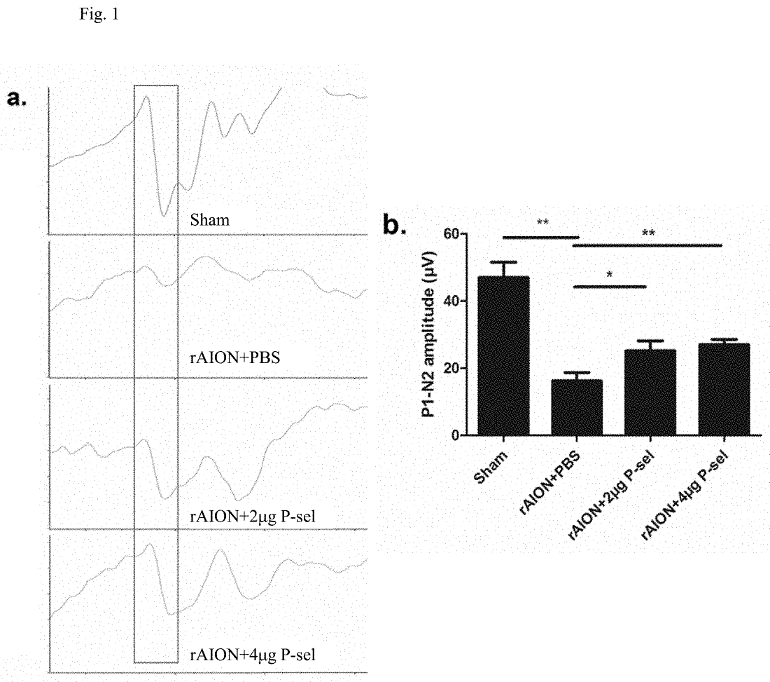

Flash visually evoked potentials (FVEPs) were measured at day 28 post-infarct. The P1-N2 amplitudes in the sham, PBS-, 2 μg P-sel- and 4 μg P-sel-treated groups were 47.00±10.15, 16.29±5.5, 25.16±7.9 and 27.02±3.4 μV, respectively. The P1-N2 amplitude was significantly preserved (FIG. 1; 2 μg P-sel, P=0.05; 4 μg P-sel, P=0.008) in both treatment groups. These data suggest that P-sel-IgG can preserve visual function in the rAION model.

### P-Sel-IgG Treatment Increased the RGC Survival Rate:

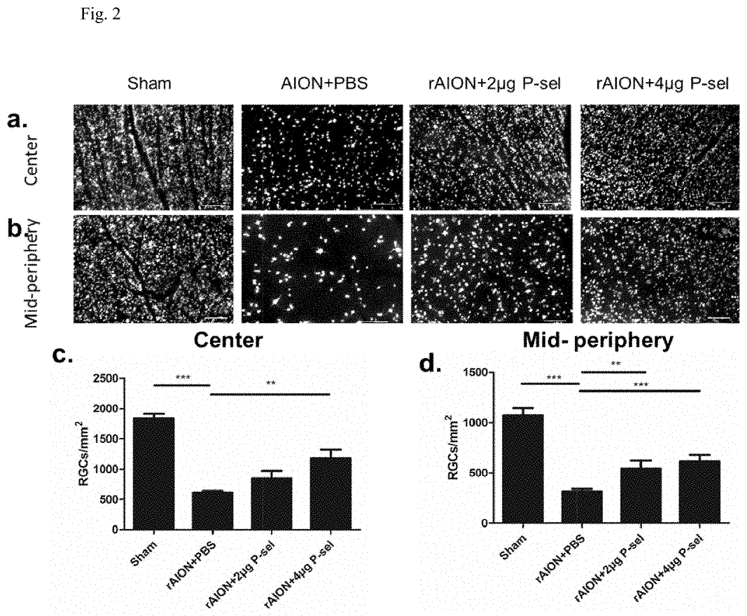

To validate the FVEP outcomes, retrograde tracing of RGCs was performed to calculate the RGC density at day 28 post-infarct. The RGC densities of the sham, PBS-, 2 μg P-sel-, and 4 μg P-sel-treated groups in the central retina were 1841±139, 612±31, 825±365, and 1009±177 cells/mm², respectively. The RGC densities of the sham, PBS-, 2 μg P-sel-, and 4 μg P-sel-treated groups in the midperipheral retina were 1063±92, 323±93, 544±66, and 614±99 cells/mm², respectively. The survival rates of RGCs in the central retina were 33.2%, 44.8%, and 54.8% in the PBS-, 2 μg P-sel-, and 4 μg P-sel-treated groups, respectively. The survival rates of RGCs in the mid-peripheral retina were 30.5%, 51.1%, and 57.7% in the PBS-, 2 μg P-sel-, and 4 μg P-sel-treated groups, respectively. There was a significant increase in RGC density between the 4 P-sel- and PBS-treated groups in both the central (FIG. 2 (a and d); P=0.002) and mid-peripheral (FIG. 2 (b and c); P=0.006) retina. However, the RGC density in the 2 μg P-sel-treated group was significantly increased only in the mid-peripheral retina (FIG. 2 (d); P=0.009), suggesting a dose-dependent effect. Together, these results validated the FVEP data and showed that P-sel-IgG treatment increased the survival rate of RGCs in a dose-dependent manner.

P-Sel Treatment Rescued RGCs from Apoptosis:

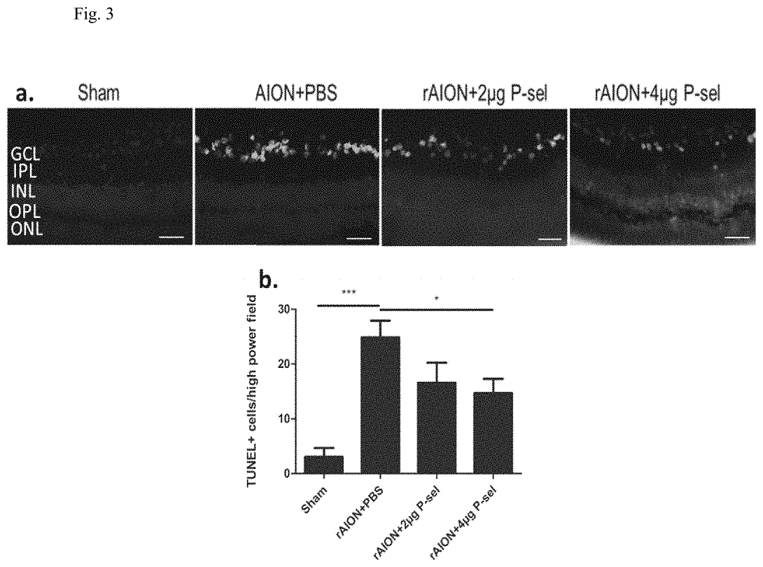

To check whether P-sel-IgG can rescue RGCs from apoptosis, an in situ TUNEL assay on retinal cross-sections was performed. The numbers of TUNEL⁺ cells in the sham, PBS-, 2 μg P-sel-, and 4 μg P-sel-treated groups were 3±2, 24±8, 16±4, and 13±6, respectively. 4 μg P-sel-treated group compared with the number in the PBS-treated group, but there was no significant difference between the PBS- and 2 μg P-sel-treated groups (FIG. 3 (a and b); P=0.01), further suggesting a dose-dependent effect. This result showed that P-sel-IgG treatment could rescue RGCs from undergoing apoptosis.

### P-Sel Prevented Blood-Borne Macrophage Infiltration in ON Tissue:

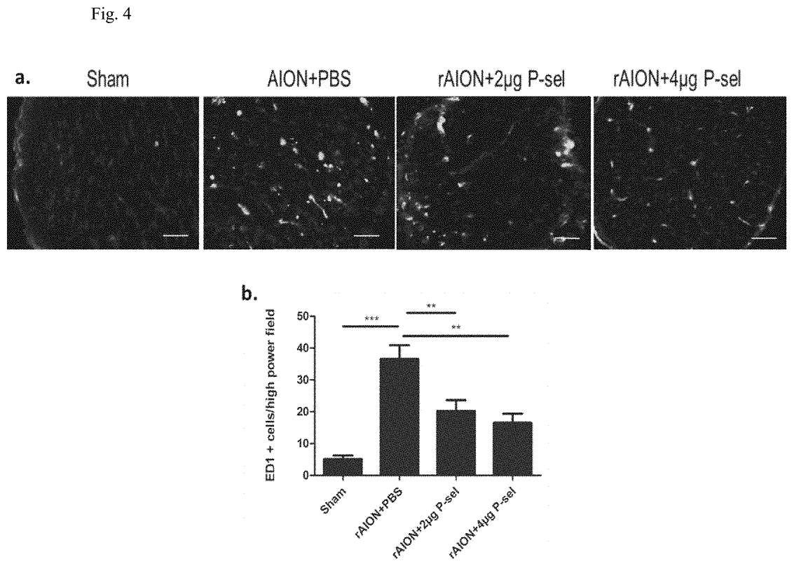

Blood-Borne macrophage infiltration into ON tissue is considered a primary response to tissue inflammation after AION. Hence, immunostaining for ED1 in ON tissue was performed to determine whether P-sel treatment could reduce blood-borne macrophage infiltration. ED1 immunostaining was performed at day 28 post-infarct. The numbers of ED1-positive cells in the sham, PBS-, 2 μg P-sel-, and 4 μg P-sel-treated groups were 5±4, 36±11, 20±10, and 16±10, respectively. There was a significant reduction in ED1-positive cells in the 2 μg P-sel- and 4 μg P-sel-treated groups (FIG. 4 (a and b); 2 μg P-sel, P=0.008; 4 μg P-sel, P=0.002). These results showed that P-sel-IgG treatment could reduce blood-borne macrophage infiltration in rAION ON tissue.

### OCT Revealed a Reduction in ON Edema and Preserved Retinal Nerve Fiber Layer (RNFL) Thickness by P-Sel Treatment:

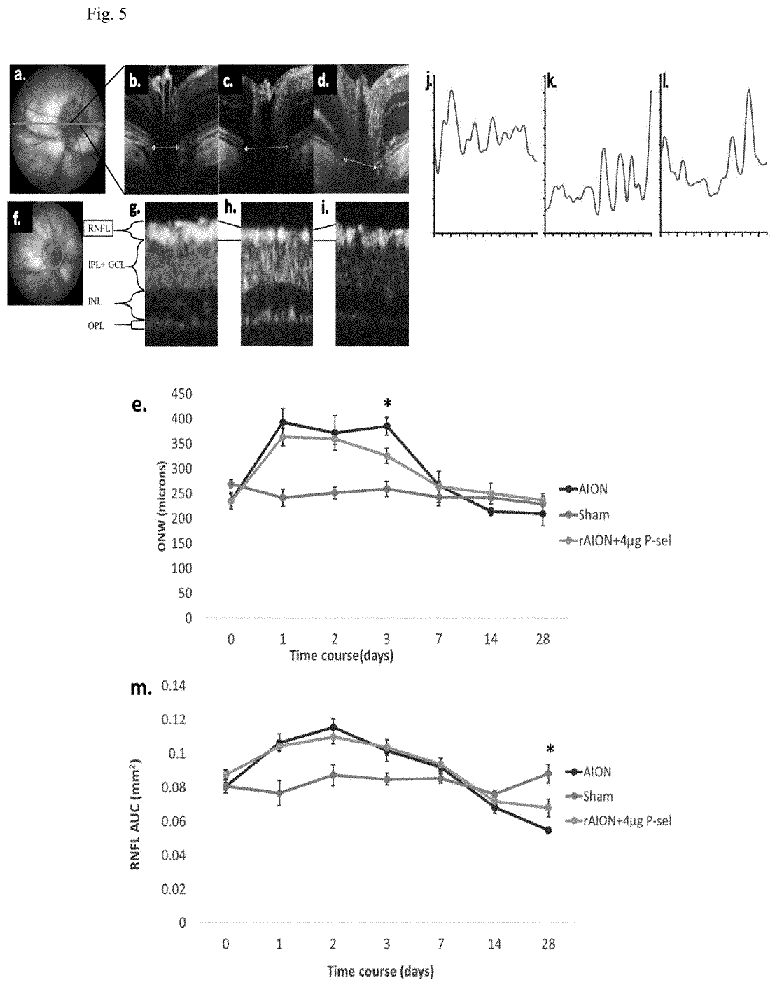

In a previous report, it was showed that the acute phase of rAION involved inflammation in ON tissue, possibly caused by a large amount of macrophage infiltration (Wen Y T, Huang T L, Huang S P, Chang C H, Tsai R K. Early applications of granulocyte colony-stimulating factor (G-CSF) can stabilize the blood-optic-nerve barrier and ameliorate inflammation in a rat model of anterior ischemic optic neuropathy (rAION). Dis Model Mech 2016; 9: 1193-1202), which potentially caused ON edema in the acute phase. In the previous experiment, the 4 μg P-sel-treated group showed more promising results and was thus chosen for further experiments. ON edema occurred immediately after AION induction; severe edema was observed at day 1 and completely recovered at day 7 (FIG. 5 (e), Table 3).

TABLE 3
ONW in time course.

| | | | rAION + | p |
|---|---|---|---|---|
| Time course | Sham | rAION + PBS | 4 μg P-sel | value |
| Day 0 | 269.5 ± 19.0 | 236 ± 40.3 | 235 ± 40.6 | n.s |
| (pre-rAION) | | | | |
| Day 1 | 242.16 ± 40 | 393.6 ± 71.2 | 363.5 ± 43.8 | n.s |
| Day 2 | 251.16 ± 28.5 | 371.8 ± 94.7 | 360.33 ± 28.1 | n.s |
| Day 3 | 259.5 ± 37.6 | 385.25 ± 43.2 | 325.5 ± 37.4 | 0.041 |
| Day 7 | 242.8 ± 37.4 | 266 ± 21.7 | 263.83 ± 74.4 | n.s |
| Day 14 | 241.4 ± 24.7 | 214.25 ± 19.9 | 250.17 ± 50.7 | n.s |
| Day 28 | 229.16 ± 38.8 | 209.75 ± 61.59 | 237.33 ± 30.1 | n.s |

Data represented as meand ± SD; unit micron; n = 6 (refered to FIG. 5)

We assumed that P-sel-IgG could reduce ON edema earlier in the course of rAION. Spectral domain OCT was used to monitor optic nerve width (ONW) over time. There was a significant reduction in ON edema at day 3 in the 4 μg P-sel-treated group (FIG. 5 (b-e); P=0.041) compared with edema in the PBS-treated group. Additionally, RNFL thickness was monitored over time. An increase in RNFL thickness was observed until day 3 due to ON edema (FIG. 5 (m), Table 4).

TABLE 4
Time course data for RNFL thickness in time course.

| | | | rAION + | p |
|---|---|---|---|---|
| Time course | Sham | rAION + PBS | 4 μg P-sel | value |
| Day 0 | 0.080 ± 0.009 | 0.082 ± 0.008 | 0.087 ± 0.009 | n.s |
| (pre-rAION) | | | | |
| Day 1 | 0.0763 ± 0.002 | 0.106 ± 0.015 | 0.104 ± 0.084 | n.s |
| Day 2 | 0.087 ± 0.013 | 0.12 ± 0.015 | 0.11 ± 0.013 | n.s |
| Day 3 | 0.0848 ± 0.0076 | 0.096 ± 0.0189 | 0.108 ± 0.014 | n.s |
| Day 7 | 0.0865 ± 0.006 | 0.092 ± 0.009 | 0.0934 ± 0.0101 | n.s |
| Day 14 | 0.0759 ± 0.005 | 0.0684 ± 0.008 | 0.0759 ± 0.00550 | n.s |
| Day 28 | 0.0857 ± 0.0122 | 0.0547 ± 0.00497 | 0.0679 ± 0.0174 | 0.0175 |

Data represented as mean ± SD; unit mm2; n = 6 (refered to FIG. 5)

RNFL thickness in the chronic phase (day 14 and day 28) indicated that the change in thickness due to ON edema was completely reduced at day 7 in all groups with rAION. Hence, any changes in RNFL thickness after complete ON edema recovery was exclusively due to 4 μg P-sel or PBS treatment. There was no significant reduction in ON edema in the 4 μg P-sel-treated group. However, RNFL thickness was significantly preserved in the 4 μg P-sel-treated group (FIG. 5 (i, l, m); P=0.017) compared with RNFL thickness in the PBS-treated group at day 28. Together, these data suggested that P-sel-IgG could reduce edema in the acute phase and preserve RNFL thickness in the chronic phase.

P-Sel-IgG Treatment Stabilizes the BONB in the Acute Phase of rAION:

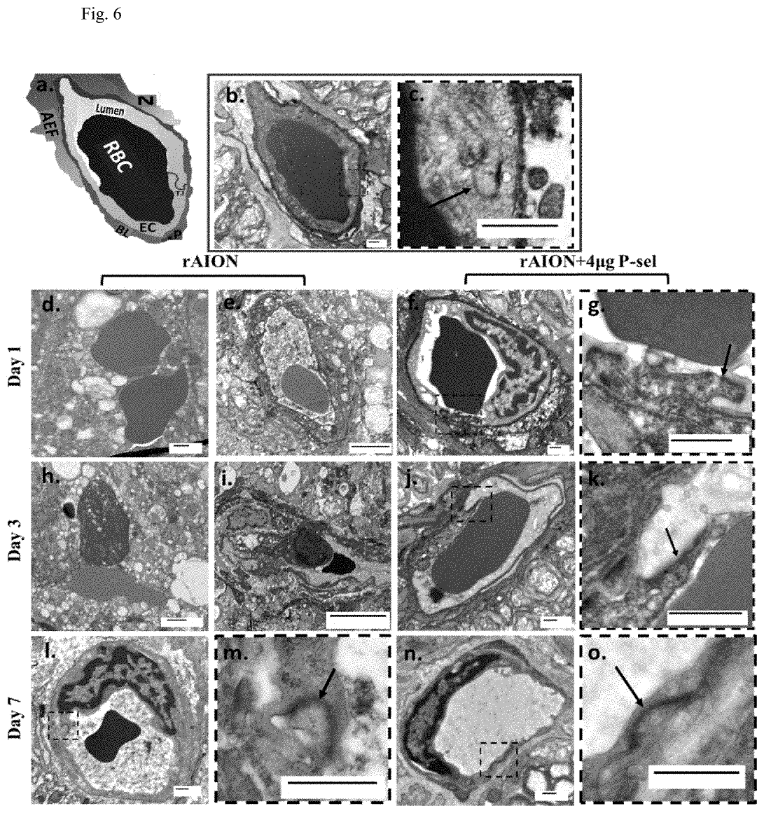

rAION caused endothelial cell damage and increased vascular permeability. Therefore, we decided to perform transmission electron microscopy (TEM) to study changes in ON tissue. Based on the OCT results (FIG. 5 (e)), we limited our study to ultrastructural changes in the acute phase (until day 7). A sham ON was used to compare ON ultrastructure. All the ultrastructures of the capillaries were clearly visible (FIG. 6 (b and c)) in the sham ON. These capillaries in the ON acted as the BONB. TEM revealed severe ultrastructural defects in the ONs of the PBS-treated group at day 1. The basal lamina was completely ruptured, and key components of the BONB were missing (FIG. 6 (d)). Most capillary units were completely damaged, but some exhibited compact basal lamina with severe vacuolation, endothelial cell damage (FIG. 6 (e)) and missing tight junctions. Similar findings were observed at day 3 (FIG. 6 (h and i)), but the number of completely damaged capillaries was reduced, and capillaries with compacted basal lamina were observed more often, indicating the transition state in the reconstitution of the BONB. When the 4 μg P-sel-treated group was examined, dramatic protection from rAION injury was observed. P-sel treatment stabilized the BONB, and the ultrastructure of the BONB was maintained at day 1 (FIG. 6 (1)) with observable tight junctions (FIG. 6 (g)). Although there was some endothelial cell damage at day 1, the tight junctions and basal lamina were still intact, and endothelial cell damage recovered at day 3. In addition, endothelial cells at day 7 in the 4 μg P-sel-treated group (FIG. 6 (n and o)) closely resembled those in the sham group, whereas endothelial cell damage was present in the PBS-treated group (FIG. 6 (i and m)) at day 7. These findings accounted for the previous OCT results (FIG. 5) in which ON edema was reduced in the 4 μg P-sel-treated group in the acute phase. This result suggested that P-sel-IgG was protective by stabilizing the BONB in the acute phase of rAION.

### P-Sel-IgG Exhibits a Nrf2-Mediated Protective Effect in the Retina:

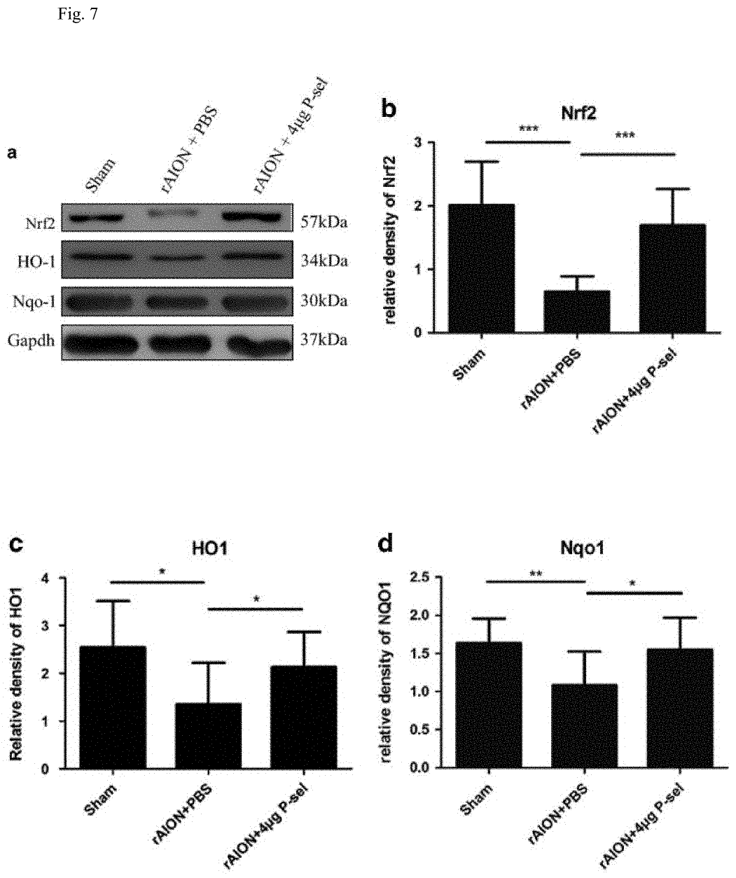

NRF2 is needed for PSGL-1-mediated protection of the liver following ischemia-reperfusion injury. PSGL-1 was a well-known ligand of P-selectin; therefore, Nrf2 and other AREs were targeted. Nrf2 expression significantly increased in the 4 μg P-sel-treated group (FIG. 7 (b)) compared with expression in the PBS-treated group. The expression levels of two AREs (Nqol and Hol) were also significantly increased in the 4 μg-P-sel-treated group (FIG. 7 (c)). This result showed that P-sel-IgG exerted neuroprotection via the Nrf2 signaling pathway.

One skilled in the art readily appreciates that the present invention is well adapted to carry out the objects and obtain the ends and advantages mentioned, as well as those inherent therein. The methods and uses thereof are representative of preferred embodiments, are exemplary, and are not intended as limitations on the scope of the invention. Modifications therein and other uses will occur to those skilled in the art. These modifications are encompassed within the spirit of the invention and are defined by the scope of the claims.

It will be readily apparent to a person skilled in the art that varying substitutions and modifications may be made to the invention disclosed herein without departing from the scope and spirit of the invention.

All patents and publications mentioned in the specification are indicative of the levels of those of ordinary skill in the art to which the invention pertains. All patents and publications are herein incorporated by reference to the same extent as if each individual publication was specifically and individually indicated to be incorporated by reference.

The invention illustratively described herein suitably may be practiced in the absence of any element or elements, limitation or limitations, which are not specifically disclosed herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

What is claimed is:

1. A method for providing ocular neuroprotection or for preventing, treating or alleviating the effects of, an ocular disease associated with retinal ganglion cell death in a subject in need thereof, comprising administering to said subject an effective amount of:
a) a recombinant P-selectin immunoglobin G (P-sel-IgG) chimeric fusion protein; or
b) a composition comprising the protein and a pharmaceutically acceptable adjuvant, vehicle, or carrier.

2. The method of claim 1, wherein the ocular disease comprises visual field loss.

3. The method of claim 1, wherein the ocular disease comprises neurodegeneration, increased intraocular pressure, an ischemic event or optic nerve injury.

4. The method of claim 3, wherein the ocular disease comprises injury to the retina or optic nerve injury.

5. The method of claim 4, wherein the injury to the retina or optic nerve injury comprises ischemia or hypoxia injury.

6. The method of claim 1, wherein the ocular disease is selected from the group consisting of glaucoma, diabetic retinopathy (DR), diabetic macular edema (DME), age related macular degeneration (AMD), Leber's hereditary optic neuropathy (LHON), Leber optic atrophy, optic neuritis, retinal artery occlusion, central retinal vein occlusion, brunch retinal vein occlusion, ischemic optic neuropathy, optic nerve injury, retinopathy of prematurity (ROP) or retinitis pigmentosa (RP), retinal ganglion degeneration, macular degeneration, hereditary optic neuropathy, metabolic optic neuropathy, optic neuropathy due to a toxic agent, neuropathy caused by adverse drug reactions or vitamin deficiency, and vision loss associated with a tumor.

7. The method of claim 6, wherein the ocular disease is ischemic optic neuropathy.

8. The method of claim 7, wherein the ischemic optic neuropathy is anterior ischemic optic neuropathy (AION).

9. The method of claim 1, wherein the ocular neuroprotection comprises neuroprotection of the optic nerve.

10. The method of claim 1, wherein the protein or the composition comprising the protein is administered as a cream, a foam, a paste, an ointment, an emulsion, a liquid solution, an eye drop, a gel, spray, a suspension, a microemulsion, microspheres, microcapsules, nanospheres, nanoparticles, lipid vesicles, liposomes, polymeric vesicles, a patch, or a contact lens.

11. The method of claim 10, wherein the protein or the composition comprising the protein is administered as a liquid solution.

12. The method of claim 11, wherein the liquid solution is administered by intravitreal injection.

13. The method of claim 1, wherein the protein comprises a C-type lectin domain and an EGF-like domain of P-selectin fused with the Fc region of human IgG₁ in a disulfide-linked homodimer form.
